(19) United States
(12) Patent Application Publication
(10) Pub. No.: US 2017/0215887 A1
(43) Pub. Date: Aug. 3, 2017

(54) METHOD FOR INCREASING PACKING DENSITY OF EMBOLIZATION MATERIAL AND DETACHING EMBOLIZATION MATERIAL

(71) Applicant: NeuroVASx, Inc., Excelsior, MN (US)

(72) Inventor: Steven J. Ferry, Excelsior, MN (US)

(73) Assignee: NeuroVASx, Inc., Excelsior, MN (US)

(21) Appl. No.: 15/501,025

(22) PCT Filed: Jul. 31, 2015
(86) PCT No.: PCT/US2015/043146

§ 371 Date: Feb. 1, 2017

Related U.S. Application Data
(60) Provisional application No. 62/031,919, filed on Aug. 1, 2014.

(51) Int. Cl. A61B 17/12

(52) U.S. Cl. CPC A61B 17/12113; A61B 17/1214; A61B 17/12022; A61B 17/12; A61B 2017/12054; A61B 2090/037

(57) ABSTRACT

Embodiments disclosed herein include an embolization material. The embolication material includes a tubular strand, with an outer surface, having a distal end and a proximal end, wherein the outer surface defines one or more slits.

### FIELD

Inventive aspects disclosed herein relate to flexible embolization filler material embodiments and to methods for treating cerebral aneurysms using the embolization filler material embodiments.

### BACKGROUND

Existing methods of detaching platinum coils use an electrical induction method. However, detachment using electrical induction is unreliable and can cause problems for a treating physician. If an embolization coil cannot be removed from a pusher mechanism after repeated attempts, the physician must remove the coil from the embolization site and use a new embolization coil. Removal of the coil from the patient can pose risks for the patient.

### IN THE DRAWINGS

FIG. 1 illustrates a side view of embolization filler material having a tubular shape with a distal bumper and oblique slits.

FIG. 2 illustrates a side view of a detacher for detaching the filler material into an aneurysm.

FIG. 3 illustrates a side view of the detacher during deployment of the embolization filler material.

### SUMMARY

One embodiment of the invention is a detacher for detaching embolization filler material having a flexible, annular, tubular configuration and an outer surface, with a proximal end and a distal end. The detacher includes a cannula, having an annulus, insertable into the proximal end of the embolization filler material annulus;

a heat bond that bonds the cannula and embolization filler material to each other at the proximal end of the embolization filler material; a buttress tube positioned and slidable over the cannula and abutted against the surface of the embolization material; a rail wire positioned within the annulus of the cannula and advanced to the distal end of the embolization filler material; a locking knob attached to the buttress tube and engaging the cannula, wherein the cannula and buttress tube act as a pusher to move the embolization material through the delivery catheter into an embolization site.

Another embodiment includes an embolization material that includes a tubular strand, with an outer surface, having a distal end and a proximal end, wherein the outer surface defines one or more slits.

### DETAILED DESCRIPTION

The following detailed description includes references to embodiments, which are described in enough detail to enable those skilled in the art to practice the invention. The embodiments may be combined, other embodiments may be utilized, or structural, and logical changes may be made without departing from the scope of the present invention. The following detailed description is, therefore, not to be taken in a limiting sense, and the scope of the inventive subject matter is defined by the appended claims and their equivalents.

In this document, the terms “a” or “an” are used to include one or more than one and the term “or” is used to refer to a nonexclusive “or” unless otherwise indicated. In addition, it is to be understood that the phraseology or terminology employed herein, and not otherwise defined, is for the purpose of description only and not of limitation. Furthermore, all publications, patents, and patent documents referred to in this document are incorporated by reference herein in their entirety, as though individually incorporated by reference. In the event of inconsistent usages between this document and those documents so incorporated by reference, the usage in the incorporated reference should be considered supplementary to that of this document; for irreconcilable inconsistencies, the usage in this document controls.

Some embodiments for embolization filler material and some method embodiments for using the embolization filler material are disclosed in US Pat. No. 6,312,421, which is incorporated herein, in its entirety, by reference. Other patents and published patent applications incorporated in their entirety by reference include the following: US20090299448; WO2009002412A1; WO2009002413A1; WO2009002403A1; US20080319533A1.

Key elements of successful embolization include filling and successful detachment of embolization material within an aneurysm or embolization site. Inventive embodiments disclosed herein address improved filling profiles as well as consistent detachment of embolization filler material embolization material.

### Overview:

Inventive embolization material embodiments disclosed herein include slits to provide improved packing within an aneurysm or other embolization site. The slits are produced on an embolization material in a variety of ways.

Mechanical detachment of the embolization material utilizes two tubes, one tube, an inner tube, slid inside the second tube, an outer tube. The inner tube is called the cannula and the outer tube is the buttress tube. The cannula tube is textured for some embodiments to increase surface area or, for other embodiments, is left smooth. The cannula is inserted within the proximal end of the embolization material and the two, cannula and embolization material are heat bonded. The buttress tube is slid over the cannula and butted against the embolization material wall.

A rail wire is placed through the center of the cannula and advanced to a distal tip of the embolization material. The rail wire supports the embolization material during delivery. A locking knob is attached to the buttress tube and engages the cannula. This locking knob prevents accidental detachment. The cannula and buttress tubes act as a pusher for the embolization material to move it through the delivery catheter and into the embolization site. When detachment is required, the rail wire is withdrawn and the locking knob is rotated counter clockwise. The cannula is grasped while the buttress tube is held stationary against the embolization material. The cannula is then pulled gently back while the outer tube buttresses the embolization material allowing the shear force from the cannula to break the seal between the cannula and the inner wall of the embolization material. The cannula and buttress tube are removed from a delivery catheter. The process is repeated with subsequent embolization material strands if needed.

### Side Wall Slitting:

The embolization material is a flexible, polymeric, tubular construct which performs in a manner similar to a wound platinum coil. The embolization filler material has an inner diameter and an outer diameter. In a first embodiment, the outer diameter of the filler material is a continuous tube.

Although the embolization material lays well into an aneurysm or other such filling location, filling is enhanced by including a series of small slits into the side wall, the slits providing additional flexibility relative to the ability of the embolization filler material to follow the contour and minimize radial force within the target embolization site as it is being filled.

Said slits can take on several configurations relative to the longitudinal aspect of the surface of the embolization filler material. In one embodiment, the slits are oblique relative to the surface of the embolization filler material and are 0.5 mm in length and spaced every 0.5 mm on center, running the proximal side of the bumper tip to the end of the embolization filler material filler strand.

Other slit configurations and placements include perpendicular, longitudinal, helical to name a few. Spacing ranges from 1 mm< to multiple millimeters and can be continuous or alternating. This can include using a combination of different slit patterns. Slits are formed by blade cutting, laser or other similar method which provides for precise slit width, length and spacing.

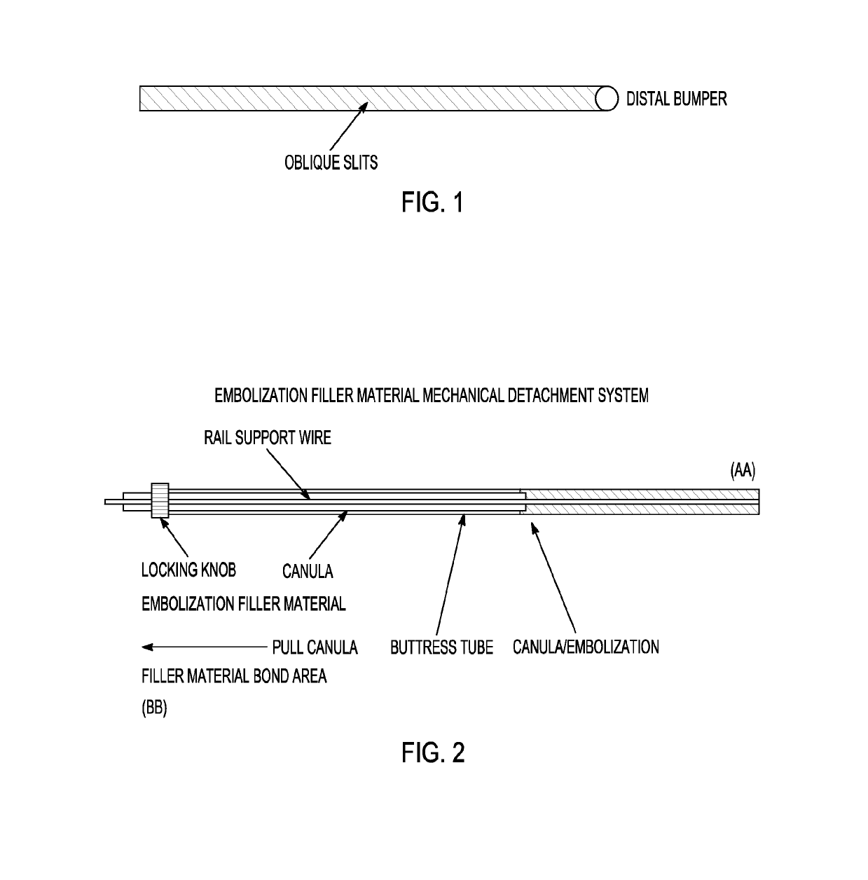

FIG. 1 below provides an example of oblique slits in the side wall of embolization filler material strand. Slits can be made on one side of the embolization filler material embolization material or on opposing sides, or at varied distances around the circumference of the filler. The embolization filler material strand terminates on its distal end in a bumper.

### Mechanical Detachment:

Another feature of the method for using the embolization filler material and embolization device is a method of detaching the embolization material from a pusher once the embolization filler material has been successfully placed using a delivery catheter. One prior art embolization filler material detachment system calls for a complex guide wire equipped with an induction coil which heats the polymeric filler and when the wire and pusher are pulled back, the filler separates from the pusher assembly. This is a prior art device that is reliable, however, as with detachment systems for Platinum embolization coils, the prior art device is not 100% effective.

The method of detaching the embolization filler material from the pusher includes a mechanical method wherein a hollow tube or cannula composed of a metal or polymer is treated or untreated. Treatment on the distal outer diameter (OD), provides added surface area. The cannula is then be inserted into the lumen of the embolization filler material for a described distance. The embolization filler material tube assembly is then connected. A variety of methods are employed to achieve this bond such as heat bonding, adhesive bonding, or reflowing to name a few. In one embodiment, the embolization filler material is heat bonded to the tube by controlling the temperature and dwell time (the time the bonding jaws are in contact with the embolization filler material surface), a bond of a specific strength is achieved. Once the cannula/embolization filler material bond is completed, a second outer tube, again a metal or polymer, is placed over the cannula. The inner diameter (ID)/OD of the respective tubes will provide for a “Slip-fit” of the two tubes. The cannula is slightly longer than the outer, or buttress tube. The buttress tube has an ID/OD, which when slid forward and engages the wall of the embolization filler material is the same as that of the embolization filler material. The buttress has a locking mechanism on it so that cannula detachment cannot be performed prematurely.

A rail wire is inserted into the cannula and advanced to the distal end of the embolization filler material to provide support as the material is being placed into the embolization site. When detachment is required, the locking mechanism is released by turning the locking knob counter clockwise and while holding the buttress tube, the cannula is drawn back. The shear force applied to the bond between the cannula and the ID of the embolization filler material allows the cannula to release at a desired force. The buttress tube engages the wall of the embolization filler material, preventing it from moving and allowing the cannula to release. Once released, the cannula and buttress tubes are removed from the delivery catheter, leaving the proximal end of the embolization filler material within the embolization site. If required, a new embolization filler material strand can be placed until adequate packing density has been achieved. It should be noted that this method could also be used with conventional platinum or other precious metal embolization coil.

### Features:

The use of slits in a side wall of the embolization filler material enables the extrusion to act more like a spring and enables it to lay into an embolization site with greater ease while minimizing radial pressure to the wall of an embolization site. Given the design of the slitting pattern, the embolization filler material does not require a tether as do other embolization devices such as platinum coils.

The mechanical detachment system exploits the polymeric nature of the embolization filler material and the ability to heat set that to a metal or polymeric cannula inserted within an embolization filler material lumen. Detachment is made simple and repeatable by utilizing a buttress that prevents the embolization filler material from moving when the cannula which is affixed to the embolization filler material, is withdrawn whereby applying a shearing force to the bond which yields a prescribed force based on the time and temperature of the formation of the bond. This system can solve detachment reliability issues currently faced using electrical detachment systems for embolization devices in medical procedures where embolization is required.

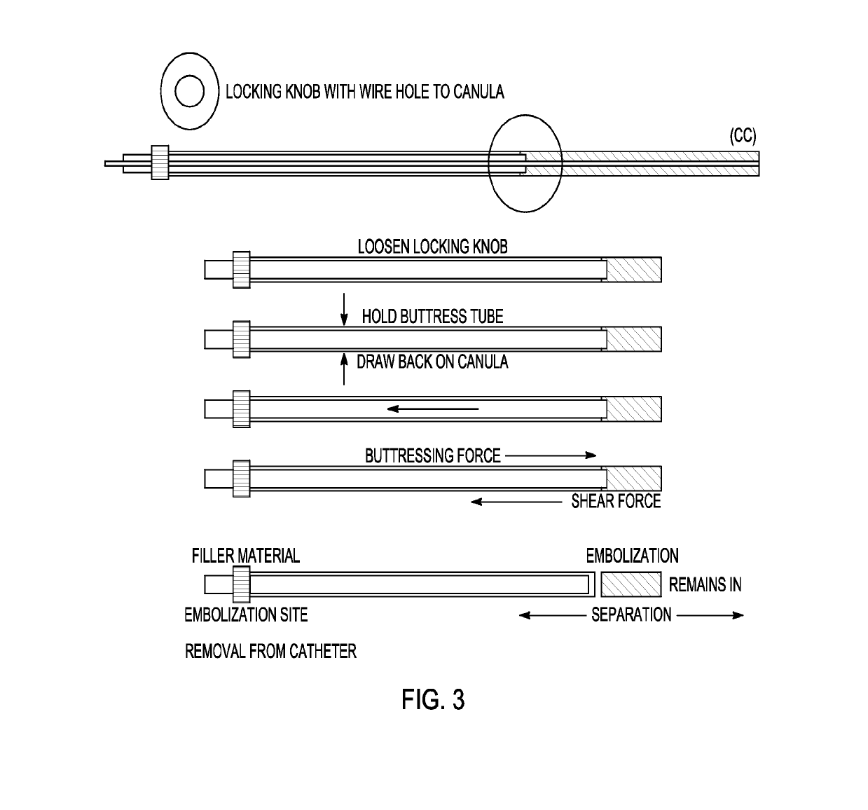

FIG. 2 illustrates the mechanical detachment approach. FIG. 3 below shows the detachment process.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the present description

What is claimed is:

1. A detacher for detaching embolization filler material having a flexible, annular, tubular configuration and an outer surface, with a proximal end and a distal end, comprising:
a cannula, having an annulus, insertable into the proximal end of the embolization filler material annulus;
a heat bond that bonds the cannula and embolization filler material to each other at the proximal end of the embolization filler material;
a buttress tube positioned and slidable over the cannula and abutted against the surface of the embolization material;
a rail wire positioned within the annulus of the cannula and advanced to the distal end of the embolization filler material;
a locking knob attached to the buttress tube and engaging the cannula, wherein the cannula and buttress tube act as a pusher to move the embolization material through the delivery catheter into an embolization site.

2. A delivery catheter for delivering the detacher of claim 1 and embolization filler material to an aneurysm.

3. The detacher of claim 1, wherein the embolization filler material defines one of more slits on the outer surface.

4. The detacher of claim 1, wherein the cannula comprises an outer surface that is textured.

5. The detacher of claim 1, wherein the cannula comprises an outer surface is smooth.

6. An embolization material comprising:
a tubular strand, with an outer surface, having a distal end and a proximal end, wherein the outer surface defines one or more slits.

7. The embolization material of claim 6, wherein the distal end of the tubular strand comprises a bumper.

8. The embolization material of claim 6, wherein the one or more slits are oblique.

9. The embolization material of claim 8, wherein the slits extend along the entire length of embolization material.

10. The embolization material of claim 6, wherein the slits are positioned to impart flexibility to the embolization material.

11. A method for detaching tubular embolization filler material defining a lumen, from a catheter, comprising:
bonding the tubular embolization filler material to a cannula;
positioning a buttress tube, having a locking mechanism over the cannula so that when the buttress tube is moved distally, the buttress tube contacts the tubular embolization filler material;
inserting a rail wire into the cannula and advancing the rail wire to a distal end of the embolization filler material;
lock the buttress tube to the cannula and embolization filler material with the locking mechanism;
advancing the buttress tube, cannula and embolization filler material to an aneurysm site with a delivery catheter; and
unlock the locking mechanism, and move the cannula proximately while holding the buttress tube stationary until the embolization filler material is released into the aneurysm site.
